(19) United States
(12) Patent Application Publication
(10) Pub. No.: US 2021/0386880 A1
(43) Pub. Date: Dec. 16, 2021

(54) MASS DECONTAMINATION SYSTEM

(71) Applicant: SOMD Loves You, Inc., California, MD (US)

(72) Inventors: Alexander Edward Scott, Lusby, MD (US); Matthew Brendan Hayes, Leonardtown, MD (US)

(73) Assignee: SOMD Loves You, Inc., California, MD (US)

(21) Appl. No.: 17/344,215

(22) Filed: Jun. 10, 2021

Related U.S. Application Data
(60) Provisional application No. 63/037,400, filed on Jun. 10, 2020.

(51) Int. Cl. A61L 2/04; A61L 2/28

(52) U.S. Cl. CPC A61L 2/04; A61L 2/28; A61L 2202/122; A61L 2202/14; A61L 2202/26

(57) ABSTRACT

The disclosure relates to a mass decontamination system that utilizes dry heat.

### CROSS REFERENCE TO RELATED APPLICATIONS

This application claims benefit of U.S. Provisional Patent Application No. 63/037,400, filed Jun. 10, 2020, the entirety of which is incorporated by reference herein.

### TECHNICAL FIELD

The present disclosure relates generally to bioburden reduction systems, and more particularly, to a mass decontamination system that utilizes dry heat.

### BACKGROUND

Conventional bioburden reduction systems are designed to remove microorganisms on an object. Healthcare facilities typically employ ultraviolet (UV) germicidal irradiation or hydrogen peroxide vapor (H₂O₂ vapor) systems to decontaminate medical equipment. However, these systems are expensive and time consuming to install. Moreover, each system has drawbacks when sanitizing certain medical equipment, such as Personal Protective Equipment (PPE). For example, UV irradiation systems require UV-C lightbulbs to function; however, the shortage of UV-C lightbulbs during the COVID-19 pandemic makes the mass decontamination, via UV irradiation systems, of PPE untenable. Similarly, H₂O₂ vapor systems are not feasible to decontaminate PPE on a mass scale as these systems require H₂O₂ to function, which is also in short supply during the COVID-19 pandemic. Additionally, applying H₂O₂ to certain PPE, such as medical masks and N95 respirators, may damage the filtration layer of these masks by removing the electrostatic charge of the filtration layer.

Conventional dry heat systems, such as an electric cooker, may expose PPE to elevated air temperatures for a period of time in order to sanitize the PPE. However, such systems require the PPE to be suspended in air without contacting or approaching metal surfaces within the chamber of the system. As the temperature of the metal surfaces are much hotter than the air temperature, any electrostatic charge required by the PPE may severely decay if the PPE contacts or approaches the metal surfaces. Further, the hotter metal surfaces may damage the PPE material. Moreover, these systems cannot utilize biological indicators to validate sterilization cycles of the PPE, as the conventional biological indicators deactivate at the temperature ranges that sanitize the PPE.

### SUMMARY

In one or more embodiments, a decontamination system includes a thermally insulated container defined by a floor, roof, and walls coupled together to form a heating chamber operably coupled to a decontamination chamber. In one or more cases, the heating chamber is configured to provide air at a set temperature to the decontamination chamber, via a plenum. In one or more cases, the decontamination chamber includes at least one air duct operably coupled to the plenum and configured to distribute the air to an interior of the decontamination chamber via one or more vents. In one or more cases, the decontamination chamber includes shelving positioned within the decontamination chamber and sized to hold a plurality of objects. In one or more cases, the shelving has a non-electrically conductive material. In one or more cases, the decontamination chamber includes at least one temperature sensor configured to monitor a temperature of the volume of the interior of the decontamination chamber. In one or more cases, the decontamination system is configured to decontaminate the plurality of objects positioned on the shelving by heating the plurality of objects for a decontamination cycle that operates for a period of time at the set temperature.

In one or more embodiments, a decontamination container includes a floor, roof, and walls coupled together to form a decontamination chamber. In one or more cases, the decontamination chamber includes at least one air duct configured to receive dry heat from a heat source and to distribute the dry heat to an interior of the decontamination chamber via one or more vents. In one or more cases, the decontamination chamber includes shelving positioned within the decontamination chamber and sized to hold a plurality of objects. In one or more cases, the shelving has a non-electrically conductive material. In one or more cases, the decontamination chamber includes at least one temperature sensor configured to monitor a temperature of the volume of the interior of the decontamination chamber. In one or more cases, the decontamination container is configured to decontaminate the plurality of objects positioned on the shelving by heating the plurality of objects for a decontamination cycle that operates for a period of time at the set temperature.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 is a perspective view of an example mass decontamination system.

FIG. 2 is a perspective view of an example adjustable support of the mass decontamination system of FIG. 1.

FIG. 3 is a rear perspective view illustrating example decontamination and utility chambers of the mass decontamination system.

FIG. 4 is a rear perspective view of the utility chamber of the mass decontamination system.

FIG. 5 is a perspective view of the example decontamination chamber of the mass decontamination system.

FIG. 6 is another perspective view of the example decontamination chamber of FIG. 5 that includes example decontamination shelving.

FIG. 7 is a perspective view of an example duct of the mass decontamination system.

FIG. 8 illustrates an example air flow diagram of the example decontamination chamber.

FIG. 9 illustrates another example air flow diagram of another decontamination chamber of the mass decontamination system.

### DETAILED DESCRIPTION

The following discussion omits or only briefly describes conventional features of bioburden reduction systems that are apparent to those skilled in the art. It is noted that various embodiments are described in detail with reference to the drawings, in which like reference numerals represent like parts and assemblies throughout the several views. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest reasonable interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively or operably connected” is such an attachment, coupling or connection that allows the pertinent structures to operate as intended by virtue of that relationship.

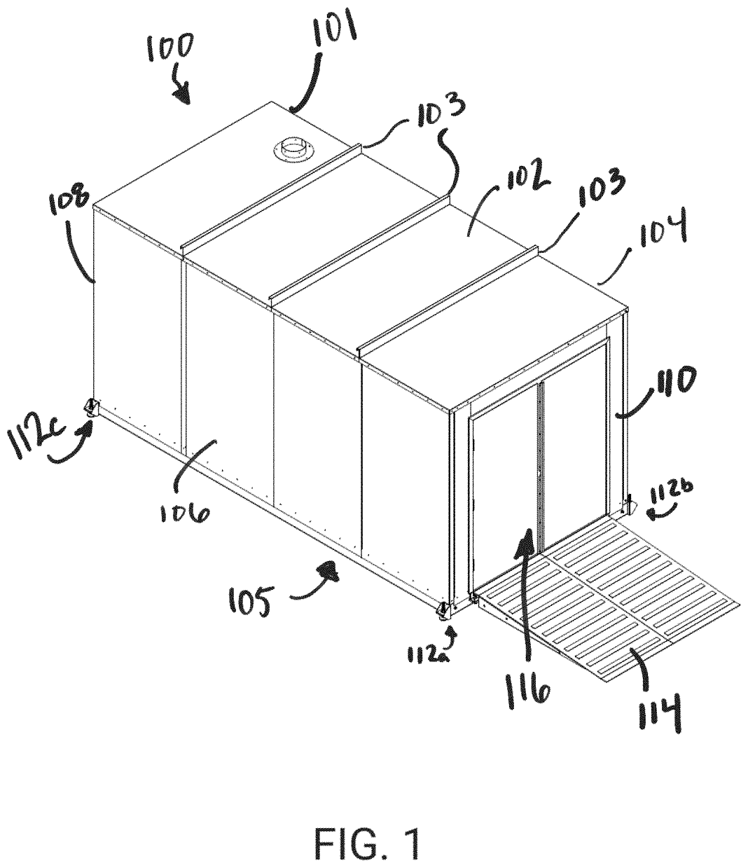

Embodiments of the present disclosure relate generally to bioburden reduction systems, and more particularly, to a mass decontamination system that utilizes dry heat. Embodiments of the mass decontamination system are described below with reference to FIGS. 1-9.

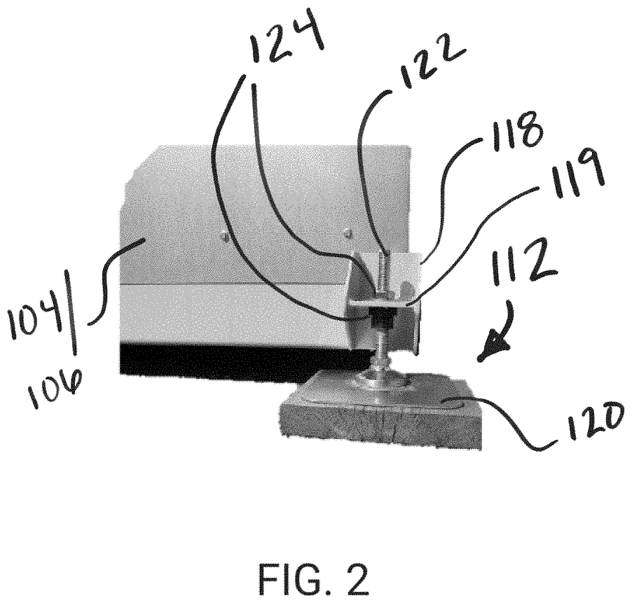

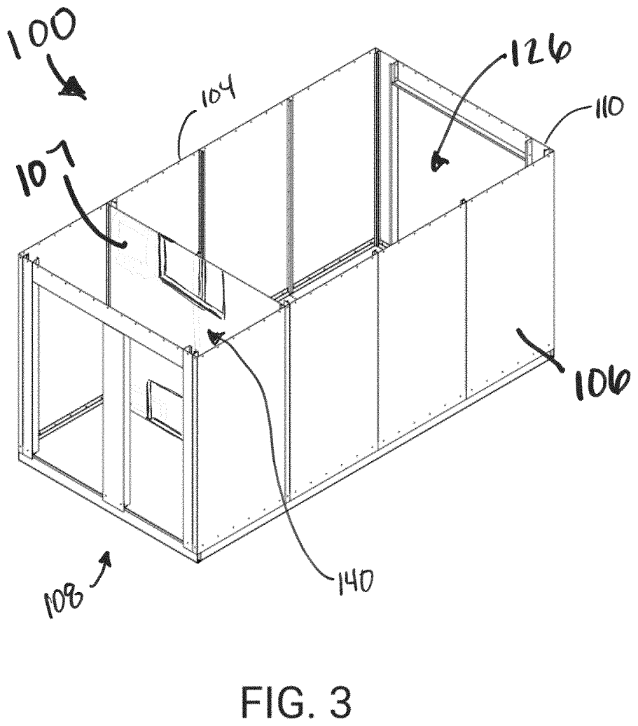

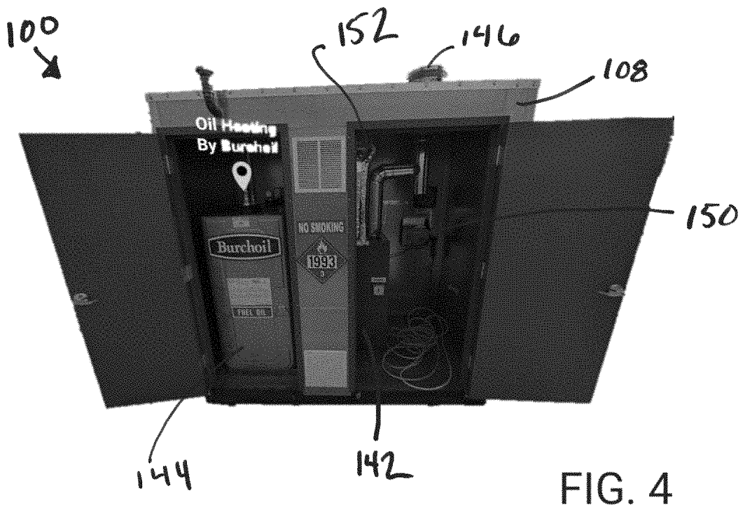

FIG. 1 is a perspective view of an example mass decontamination system 100 (hereinafter “system 100”). FIG. 2 is a perspective view of an example adjustable support 112 of the mass decontamination system 100 of FIG. 1. FIG. 3 is a rear perspective view illustrating example decontamination chamber 126 and utility chamber 140 of the mass decontamination system 100. FIG. 4 is a rear perspective view of the utility chamber 140 of the mass decontamination system 100.

In one or more embodiments, the system 100 is an enclosed container 101 defined by side walls 104, 106, 108, and 110, roof 102, and floor 105. In one or more cases, the side walls 104, 106, 108, and 110, roof 102, and floor 105 are formed from steel, wood, or other like material that retains heat. For the cases in which the side walls 104, 106, 108, and 110, roof 102, and floor 105 are formed from steel, the side walls 104, 106, 108, and 110, roof 102, and floor 105 may be coupled together via, for example, but not limited to, welding, riveting, bolting together, or any combination thereof, the side walls 104, 106, 108, and 110, roof 102, and floor 105. Supporting joists 103 may extend across the roof 102 from opposing walls, such as wall 106 and wall 104, and be configured to strengthen the shape of the container 101.

In one or more cases, the system 100 includes a plurality of adjustable supports 112, such as adjustable supports 112a, 112b, 112c, and 112d, disposed around the floor 105. The adjustable supports 112a, 112b, 112c, and 112d are configured to be individually raised or lowered to level the container 101. In one or more cases, the adjustable support 112 includes a mount 118 fastened to the container 101, and an elongated rod 122 passing through a mounting plate 119 of the mount 118. A rigid and flat base 120 may be disposed on a lower end of the rod 122. One or two fasteners, such as fasteners 124, may be disposed over the rod 122 and on opposite sides of the mounting plate 119. The fasteners 124 may be fastened towards one another, such that the fasteners 124 sandwich the mounting plate 119 along the length of the rod 122 and fasten the mount 118 to the rod 122.

In one or more cases, wall 110 includes one or more doors 116 to access the decontamination chamber 126, and wall 108 includes one or more doors to access the utility chamber 140. In some cases, a ramp 114 may be removably coupled to the wall 110 and provide access for a user to easily roll, for example, a cart from outside the container 101 to inside the container 101 and onto the floor 105.

In one or more cases, the system 100 includes a dividing wall 107 therein, which separates the decontamination chamber 126 and the utility chamber 140. In one or more other cases, the container 101 of the system 100 includes the decontamination chamber 126 and the utility chamber 140 is disposed in a separate container a distance away from the decontamination chamber 126. For example, the utility chamber 140 may be included in a separate container, or may be integrated with a heating, ventilation, and air conditioning (HVAC) system of a nearby building (e.g., a HVAC system of a hospital). In one or more cases, the decontamination chamber 126 and the utility chamber 140 may be connected to one another via a plenum 152, which passes air from a furnace 142 within the utility chamber 140 and through an air duct 128 in the decontamination chamber 126. The decontamination chamber 126 and the utility chamber 140 may be connected to one another via a return 134, which passes air from the decontamination chamber 126 to the furnace 142. The dividing wall 107 include openings for the plenum 152 and the return 134.

In one or more cases, the utility chamber 140 houses a tank 144 operably coupled to the furnace 142 and a blower unit. The tank 144 may, for example, be a heating fuel tank, such as a propane or natural gas fuel tank, or a double-walled heating oil tank. The blower unit is configured to pull air from outside the container 101, via vent 146, and pass the air into the plenum 152. The furnace 142 may be an oil-burning furnace configured to heat the air passing into the plenum 152. The components may be operably coupled to one another and connected to and powered by an electrical source, such as a 120V outlet connected to a 15-amp circuit. In one or more cases, the furnace 142 does not have a line-of-sight connection to the decontamination chamber 126. Thus, the decontamination chamber 126 is not subjected to radiant heat generated by the furnace 142 and is heated by the hot air passing through the plenum 152. The air within the decontamination chamber 126 passes through the return 134 and, in some cases, to the furnace 142. The air that flows into the decontamination chamber 126 does not include additives, and therefore, the concentration of the air within the decontamination chamber 126 does not need to be monitored.

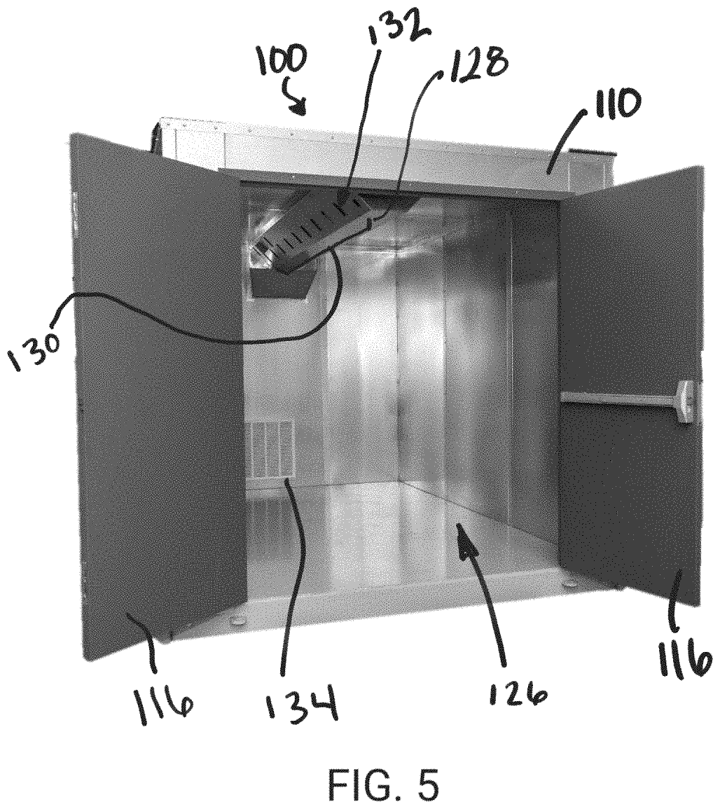

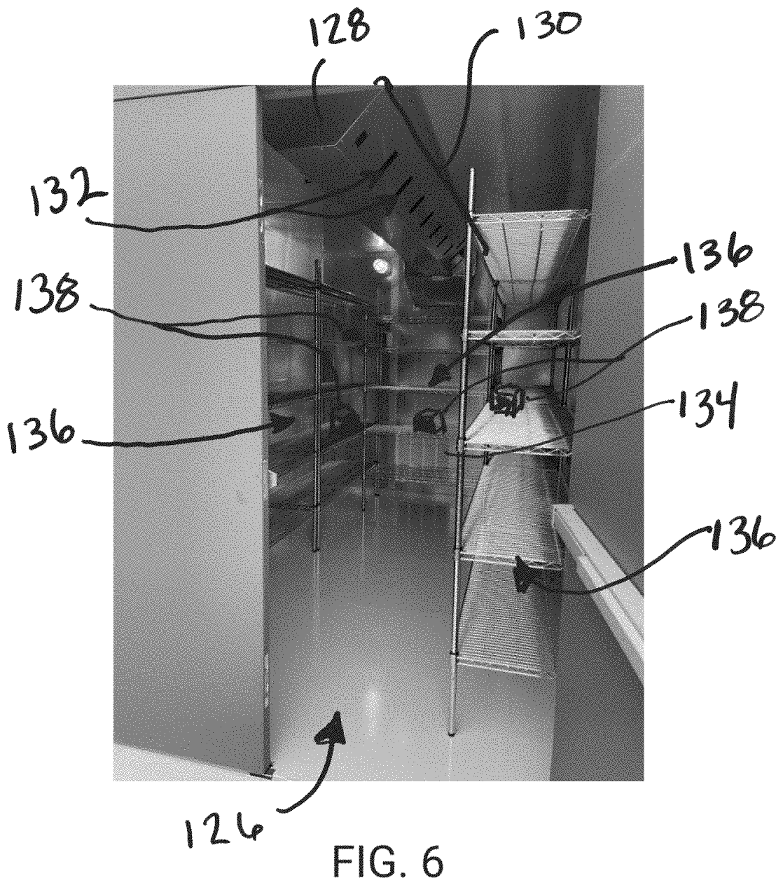

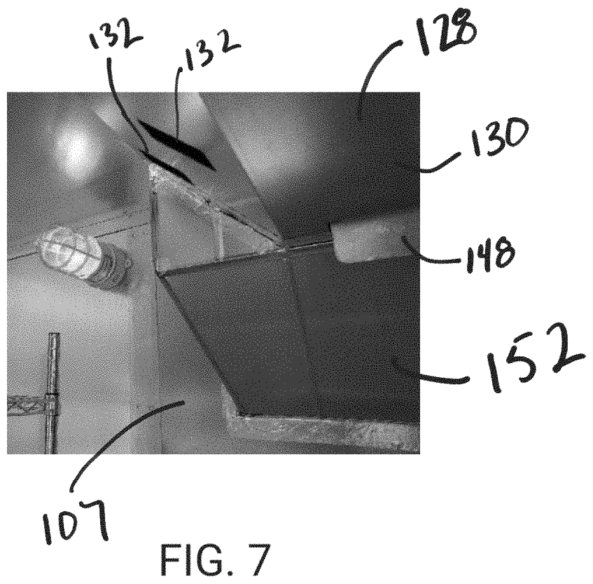

FIG. 5 is a perspective view of the decontamination chamber 126. FIG. 6 is another perspective view of the decontamination chamber 126 that includes example decontamination shelving 136. FIG. 7 is a perspective view of an example duct 128.

The decontamination chamber 126 may be sized to hold a plurality of objects, for example, but not limited to, a plurality of personal protective equipment objects. For example, the decontamination chamber 126 may be sized to hold a plurality (e.g., 2,400) of N95 respirators during a decontamination cycle. The decontamination chamber 126 houses and decontaminates the plurality of objects by subjecting the objects to increased air temperatures for a period of time. It should be noted that the examples herein discuss the system 100 sanitizing medical masks and N95 respirators; however, it should be understood that the system 100 can house and decontaminate any object and any number of objects by subjecting the object to increased air temperatures for a period of time.

The decontamination chamber 126 may include shelving 136 generally positioned near the walls 104, 106, and 107 of the container 101, such that the shelving 136 does not restrict air flow in the decontamination chamber 126. In some cases, the shelving 136 is formed from wire racks that facilitate air flow by allowing air to circulate through the wire racks. In one or more cases, the shelving 136 is made of or coated with a non-electrically conductive material to avoid an electrostatic discharge of a portion of the object, such as a filtration layer of an N95 respirator. In such cases, the objects may be placed on the shelving 136 within the decontamination chamber 126 without electrostatically discharging the object.

The plenum 152 is operably connected to the air duct 128. In one or more cases, the air duct 128 extends the length of the decontamination chamber 126. The air duct 128 may include a series of vents 130 disposed on each side of the air duct 128. A vent 132 may be formed in any shape that allows the air to pass from the air duct 128 into the area of the decontamination chamber 126. The series of vents 130 may be distributed along the length of the air duct 128 to provide even temperatures throughout the volume of the decontamination chamber 126. For example, the air duct 126 may distribute the air, such that the temperature at various areas within the volume of the chamber 126 ranges from 1° C. to 2° C. In one or more cases, a vent 132 may include a deflector 148 that is configured to direct air towards a specific location within the chamber 126. For example, deflector 148 may be configured to direct air from air duct 128 towards the dividing wall 107. A deflector 148 may be a rigid planar member that is coupled to a portion of the outer perimeter of a vent 132. The deflector 148 may be angled such that air exiting the vent 132 deflects off of the deflector 148 and is redirected to another location of the chamber 126. In some cases, the deflector 148 is formed by cutting a portion of the duct 128 to form an initial shape of the vent 132. The uncut portion of the duct 128 couples the duct 128 to the vent 132. The cut portion of the duct 128 may be bent at the uncut portion of the duct 128, thereby forming the deflector 148 as well as a cavity defining the vent 132. In other alternative cases, the deflector 148 may be attached (e.g., via fastening sheet metal) to a portion of the duct 128 (e.g., an area near a vent 132), such that the deflector 148 deflects air exiting the respective vent 132.

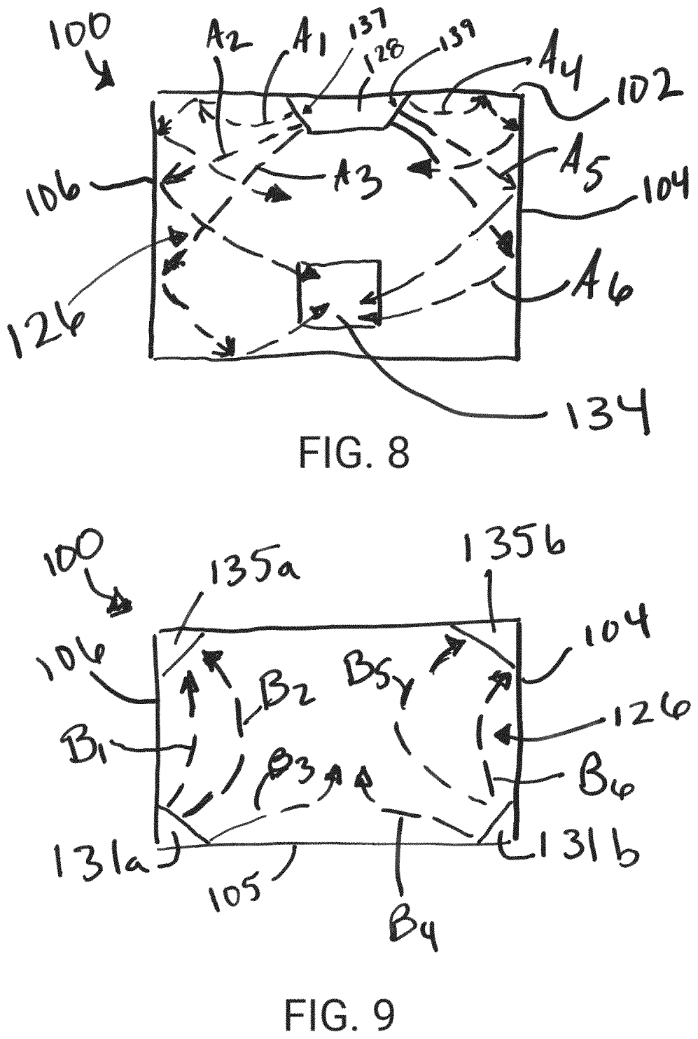

In one or more cases, the decontamination chamber 126 may include one air duct 128 that is disposed on the ceiling, i.e., the bottom surface of the roof 102, of the decontamination chamber 126. In some cases, the air duct 128 is centered or generally centered within the decontamination chamber 126. In other cases, the air duct 128 may be offset towards one wall, such as wall 104 or wall 106 of the decontamination chamber 126. For cases in which the decontamination chamber 126 includes one air duct 128, the series of vents 130 may be disposed on each side of the air duct 128 and configured to direct air away from one another and towards the adjacent wall. For example, as illustrated in FIG. 8, one series of vents 137 may direct air in directions Al, A2, and A3 towards wall 106, and another series of vents 139 may direct air in directions A4, A5, and A6 towards wall 104. The air may circulate within the decontamination chamber 126 to increase or decrease the air temperature within the decontamination chamber 126, such that the air temperature is the same or generally the same within each area of the chamber 126. For example, the utility chamber 140 may provide air with varying temperatures to the decontamination chamber 126, such that the temperature at various areas of the chamber 126 ranges from 1° C. to 2° C. The air may exit chamber 126 via the return 134. It is noted that FIG. 8 illustrates one air duct 128 disposed on the ceiling of the decontamination chamber 126, but it should be understood that multiple air ducts 128 may be disposed across the ceiling and extend in parallel along the length of the decontamination chamber 126.

In other cases, the decontamination chamber 126 may include multiple air ducts, such as air ducts 131a and 131b, that are disposed in the lower corners of the chamber 126. For example, air duct 131a may be positioned in the corner formed by wall 106 and floor 105, and air duct 131b may be positioned in the corner formed by wall 104 and floor 105. The plenum 152 may be operably connected to each air duct 131a and 13 lb. The vents 132 of the air ducts 131a and 131b may be directed to force air towards one another and towards the center of the chamber 126. For example, as illustrated in FIG. 9, the vents of air duct 131a may direct air in directions B1, B2, and B3 towards the center of chamber 126 and air duct 131b, and the vents of air duct 131b may direct air in directions B4, B5, and B6 towards the center of chamber 126 and air duct 131a. In some cases, the air ducts 131a, 131b extend an entire length or almost an entire length of the chamber 126. In one or more cases, the chamber 126 includes one or more returns, such as returns 135a and 135b, that are disposed in the upper corners of the chamber 126. For example, return 135a may be positioned in the corner formed by wall 106 and roof 102, and return 135b may be positioned in the corner formed by wall 104 and roof 102. The air may exit the chamber 126 via the returns 135a and 135b. In other cases, the chamber may include one or more returns positioned on the dividing wall 107.

In one or more cases, a temperature sensor 138 may be disposed in the chamber 126 to monitor the temperature within the volume of the chamber 126. For example, the temperature sensor 138 may be positioned on the shelving 136. In another example, the temperature sensor 138 may be fastened to a wall of the chamber 126. In some cases, the temperature sensor 138 may be operably connected to a chart plotter 150 located within the utility chamber 140. The chart plotter 150 may record the time and temperature of the temperature sensor 138. For example, the chart plotter 150 may log the temperatures for the temperature sensor 138 at regular intervals (e.g., every five seconds) within the time period (e.g., thirty minutes to sixty minutes) for a decontamination cycle. The chart plotter 150 provides an indication when the decontamination cycle completed. For example, the chart plotter 150 may indicate that the interior volume of the chamber 126 was heated to a temperature for a period of time that corresponds to a completion of the decontamination cycle. In one or more cases, during the decontamination cycle for N95 respirators, the furnace 142 is configured to regulate the temperature, within the decontamination chamber 126, between 70° C. and 75° C. for thirty to sixty minutes.

To begin the decontamination process, the decontamination chamber 126 is pre-heated to a preset temperature (e.g., 75° C.) or temperature range. In some cases, one or more objects, for example, N95 respirators, may be placed in respective mesh bags that include a label of the user of the N95 respirator, whether the N95 respirator passed a previous decontamination cycle, and/or a number of times the N95 respirator underwent a decontamination cycle. When the decontamination chamber 126 has reached the preset temperature, such as 75° C., or has reached a stable temperature range, such as between 70° C. and 82° C., the N95 respirators and mesh bags are placed on the shelving 136. In an example, the system 100 initiates the decontamination cycle by heating the N95 respirators to 75° C. −85° C. or about 75° C. to 85° C. for 60 minutes or about 60 minutes. In another example, the system 100 heats the N95 respirators to 70° C. or about 70° C. for three hours or about three hours. In some cases, the temperature within the decontamination chamber 126 may decrease when a user opens the doors 116 to enter the chamber 126 and place the mesh bags on the shelving 136. In such cases, the decontamination cycle includes the time period to return to the preset temperature or stable temperature range and the heating period, e.g., 60 minutes. In some cases, if the temperature exceeds 85° C., the N95 respirator is considered damaged and discarded.

In one or more cases, the temperature set points may be determined based on the time and/or temperature rates to disable SARS and MERS coronaviruses, the temperature at which the electrostatic charge in certain PPE is destroyed, and/or the time and/or temperature rates needed to kill other forms of potentially dangerous diseases, particularly bacteria.

In one or more other cases, a plurality of temperature sensors 138 may be disposed throughout the chamber 126 to monitor the temperatures at various points within the volume of the chamber 126. For example, the temperature sensors 138 may be positioned on the shelving 136. In another example, the temperature sensors 138 may be fastened to one or more walls of the chamber 126. In some cases, the temperature sensors 138 may be operably connected to computer system, which may include a controller unit, such as an Arduino Due, for acquiring temperature data and controlling the system and may include an interface unit, such as a Raspberry Pi, to provide a touch screen for logic, interface features, and data storage. In some cases, the controller unit receives the temperature data from the temperature sensors 138 and tracks the temperature at respective times for each temperature sensor 138. The interface unit presents a graphical display on a display device of the computer system. The graphical display may indicate whether the decontamination cycle for the PPE within the chamber 126 or within the region of a respective temperature sensor 138 is in process, complete, or failed/damaged. In one or more other cases, the controller unit may provide a signal to a visual indicator that is operably coupled to a temperature sensor 138. The signal may correspond to whether the PPE within the region of a respective temperature sensor 138 is in process, complete, or failed/damaged. The visual indicator, such as a multicolored LED, may be positioned near the corresponding temperature sensor 138 and light a certain color to indicate a decontamination cycle status, e.g., in process, complete, or failed/damaged, of the PPE.

In one or more cases, the computer system determines whether a decontamination cycle is completed based on the time and temperature set points and by monitoring the time and temperature at which the PPE is heated. For example, if the computer system determines that the temperature is below 70° C., the computer system pauses or does not initiate the timer for the decontamination cycle. In another example, if the computer system determines that the temperature is between 70° C. and 75° C., the computer system counts at a slower rate. That is, the computer system heats the PPE for a longer decontamination cycle, e.g., three hours, if the computer system determines that the temperature is between 70° C. and 75° C. In another example, if the computer system determines that the temperature is between 75° C. and 85° C., the computer system counts at a rate of one hour. That is, the computer system heats the PPE for one hour if the computer system determines that the temperature is between 75° C. and 85° C. In another example, if the computer system determines that the temperature is above 85° C., the computer system determines that the PPE in that region of the decontamination chamber 126 is damaged. The computer system may be configured to vary the heating time and temperature of the decontamination cycle based on the received temperature data.

During a decontamination cycle, as a mass decontamination system, the system 100 may heat, for example, but not limited to, 2000-2400 N95 respirators. In some cases, an N95 respirator may be reused and decontaminated several times (e.g., thirty times) before being discarded. In some cases, an N95 respirator may include a label that tracks a number of times that the N95 respirator underwent a decontamination cycle.

As used herein, the term “about” in reference to a numerical value means plus or minus 15% of the numerical value of the number with which it is being used.

Further, it is noted that the instruction manual, titled “Instructions for Healthcare Facilities and Personnel: Preparation of Compatible N95 Respirators for Bioburden Reduction and Installation and Operation of a Semi-Automated Bioburden Reduction Module for Emergency Use of Compatible N95 Respirators” and published on Jan. 13, 2021 via https://www.somdlovesyou.org/the-hot-box, is incorporated into this disclosure by reference in its entirety.

The various embodiments described above are provided by way of illustration only and should not be construed to limit the claims attached hereto. Those skilled in the art will readily recognize various modifications and changes that may be made without following the example embodiments and applications illustrated and described herein, and without departing from the spirit and scope of the following claims.

What is claimed is:

1. A decontamination system comprising:
a thermally insulated container defined by a floor, roof, and walls coupled together to form a heating chamber operably coupled to a decontamination chamber;
wherein the heating chamber is configured to provide air at a set temperature to the decontamination chamber, via a plenum;
wherein the decontamination chamber comprises:
at least one air duct operably coupled to the plenum and configured to distribute the air to an interior of the decontamination chamber via one or more vents,
shelving positioned within the decontamination chamber and sized to hold a plurality of objects, the shelving having a non-electrically conductive material, and
at least one temperature sensor configured to monitor a temperature of the volume of the interior of the decontamination chamber; and
wherein the decontamination system is configured to decontaminate the plurality of objects positioned on the shelving by heating the plurality of objects for a decontamination cycle that operates for a period of time at the set temperature.

2. The decontamination system of claim 1, wherein the provided air comprises dry heat.

3. The decontamination system of claim 1, wherein the container further comprises a plurality of adjustable supports disposed around the floor of the container, the plurality of adjustable supports being configured to level the container.

4. The decontamination system of claim 1, wherein:
the decontamination chamber comprises at least one door disposed on a wall of the container to access the interior of the decontamination chamber, and
the container further comprises an adjustable ramp aligned with the at least one door and positioned outside of the wall of the container.

5. The decontamination system of claim 1, wherein the plurality of objects comprises medical masks, N95 respirators, or a combination of medical masks and N95 respirators.

6. The decontamination system of claim 1, wherein the container further comprises at least one return disposed between the heating chamber and the decontamination chamber, the at least one return configured to receive air from the decontamination chamber and pass the air to the heating chamber.

7. The decontamination system of claim 1, wherein:
the at least one air duct is centrally positioned on a ceiling of the decontamination chamber and extends a length of the decontamination chamber, and
a first set of vents and a second set of vents disposed on opposite sides of the at least one air duct and positioned to direct the air away from one another.

8. The decontamination system of claim 1, wherein:
a first air duct and a second air duct are positioned in lower corners of the decontamination chamber and extend a first distance of the decontamination chamber, and
a first set of vents disposed on the first air duct and a second set of vents disposed on the second air duct are positioned to direct the air towards one another.

9. The decontamination system of claim 8, wherein:
a first return and a second return are positioned in upper corners of the decontamination chamber and extend a second distance of the decontamination chamber, and
the first return and the second return being configured to receive air from the decontamination chamber and pass the air to the heating chamber.

10. The decontamination system of claim 1, wherein the at least one air duct further comprises one or more deflectors positioned adjacent to a respective vent, such that the air exiting the vent is deflected in another direction.

11. The decontamination system of claim 1, wherein:
the at least one temperature senor is operably coupled to a chart plotter disposed within the heating chamber, and
the chart plotter being configured to record and log a time and respective temperature of the at least one sensor.

12. The decontamination system of claim 1, wherein the at least one temperature sensor is fastened to a wall of the container.

13. The decontamination system of claim 1, wherein the at least one temperature sensor is operably coupled to a computer system configured to provide a status update of the decontamination cycle of the objects adjacent the at least one temperature sensor, the status update being displayed on a graphical display.

14. The decontamination system of claim 1, wherein the heating chamber is configured to pre-heat the decontamination chamber to a temperature ranging between 70° C. and 82° C.

15. The decontamination system of claim 1, wherein the decontamination cycle comprises heating the decontamination chamber to a temperature ranging between 75° C. and 85° C. and for a period of time ranging between 30 and 60 minutes.

16. A decontamination container comprising:
a floor, roof, and walls coupled together to form a decontamination chamber, wherein the decontamination chamber comprises:
at least one air duct configured to receive dry heat from a heat source and to distribute the dry heat to an interior of the decontamination chamber via one or more vents,
shelving positioned within the decontamination chamber and sized to hold a plurality of objects, the shelving having a non-electrically conductive material, and
at least one temperature sensor configured to monitor a temperature of the volume of the interior of the decontamination chamber; and
wherein the decontamination container is configured to decontaminate the plurality of objects positioned on the shelving by heating the plurality of objects for a decontamination cycle that operates for a period of time at the set temperature.

17. The decontamination container of claim 16, wherein the plurality of objects comprises medical masks, N95 respirators, or a combination of medical masks and N95 respirators.

18. The decontamination container of claim 16, wherein:
the at least one air duct is centrally positioned on a ceiling of the decontamination chamber and extends a length of the decontamination chamber, and
a first set of vents and a second set of vents disposed on opposite sides of the at least one air duct and positioned to direct the air away from one another.

19. The decontamination container of claim 16, wherein:
a first air duct and a second air duct are positioned in lower corners of the decontamination chamber and extend a first distance of the decontamination chamber, and
a first set of vents disposed on the first air duct and a second set of vents disposed on the second air duct are positioned to direct the air towards one another.

20. The decontamination container of claim 16, wherein the decontamination cycle comprises heating the decontamination chamber to a temperature ranging between 75° C. and 85° C. and for a period of time ranging between 30 and 60 minutes.
